(19) United States
(12) Patent Application Publication
(10) Pub. No.: US 2023/0049297 A1
(43) Pub. Date: Feb. 16, 2023

(54) MEDICAL ARTICLE FOR THE PROPHYLAXIS OF DECUBITUS ULCERS

(71) Applicant: PAUL HARTMANN AG, Heidenheim Baden-Württemberg (DE)

(72) Inventors: Axel ECKSTEIN, Heidenheim (DE); Maja KRÄMER, Herbrechtingen (DE)

(21) Appl. No.: 17/784,272

(22) PCT Filed: Dec. 17, 2020
(86) PCT No.: PCT/EP2020/086836

§ 371 Date: Jun. 10, 2022

(30) Foreign Application Priority Data
Dec. 19, 2019 (DE) 10 2019 135 061.1

(51) Int. Cl. A61F 13/02

(52) U.S. Cl. CPC A61F 13/0206; A61F 13/0253; A61F 2013/00148

(57) ABSTRACT

The present invention relates to a medical article (10) for the prophylaxis of the formation and/or worsening of decubitus ulcers, containing a compress (12) and a skin contact layer (13), which skin contact layer can adhere to skin to be treated, wherein the compress (12) has a proximal surface and a distal surface and comprises a core (15) and a shell (14) surrounding the core (15); wherein the core (15) has a proximal surface and a distal surface and comprises a nonwoven material, which is composed of fibers and absorbent particles, wherein the compress (12) has a longitudinal direction having a first modulus of elasticity and a transverse direction having a second modulus of elasticity and the first modulus of elasticity is greater than the second modulus of elasticity.

The present invention relates to medical articles for prophylaxis of the development and/or worsening of decubitus ulcers.

Decubitus ulcer (synonyms: decubitus, decubitus sore, pressure sore/pressure ulcer, bedsore/bed ulcer) is defined as a trophic disorder of tissues (mainly skin and subcutaneous tissue) accompanied by necrosis, maceration and possibly infection, the disorder being caused by external (long-term) pressure with compression of vessels and local ischemia. Decubitus ulcers mainly occur during bed confinement, especially on parts of the body at which the skin is in direct contact with a bone, but also, for example, under ill-fitting prostheses and excessively tight plaster casts.

Decubitus is divided into the following stages. Here, stage II, stage III and stage IV decubitus ulcers are known as chronic wounds:
Stage I: This is a persistent, localized erythema that remains even when pressure is relieved. The redness is sharply defined and may be indurated or abnormally warm. The skin is still intact.
Stage II: Blistering and skin abrasion occur in this phase, resulting in partial loss of skin. There is damage to the epidermis right up to parts of the dermis. A superficial wound or a shallow ulcer is present in this phase.
Stage III: In this advanced stage, loss of all layers of the skin can already be observed. Furthermore, damage to the subcutaneous tissue and possibly necrosis that may extend up to the underlying muscle tissue can be observed. Experience has shown that the necrotic tissue must first be demarcated before the full extent of the tissue damage can be seen. Stage III decubitus presents clinically as an open, deep ulcer.
Stage IV: In this extremely critical stage, all layers of the skin are lost with extensive destruction, tissue necrosis, or damage to muscles, bones or supporting structures (tendons, joint capsules). Stage IV decubitus presents clinically as a large, open and deep ulcer.

Decubitus ulcers can develop when pressure is exerted on soft tissue, interrupting some or all of the blood flow to said tissue. Shearing forces arising from friction on specific skin areas also significantly contribute to the development of decubitus ulcers. Such shearing forces arise from movement of the skin on surfaces of other materials, for example a bed sheet. When the shearing forces that occur cause a patient’s skin to shift against the tissues below the skin, blood vessels in said tissues are also compressed, restricting or interrupting the blood flow to said tissues. Decubitus ulcers tend to develop on specific skin areas, for example on the skin covering the sacral region, coccyx, heel or hip bones. Other areas at which they commonly occur are elbows, knees, joints and shoulders. Decubitus ulcers usually develop in patients whose movement is severely restricted or completely prevented, for example in bedridden patients or those who are largely wheelchair-dependent. These patients are limited in their ability to independently move their limbs, where there are areas under pressure, for relief of pressure. Other factors may play an important role in the development of decubitus ulcers, for example protein deficiency, an unfavorable microclimate (skin moisture due to sweating, wound exudate or incontinence), vascular diseases such as atherosclerosis, or diseases that reduce skin sensitivity such as paralysis or neuropathy. The cure rate for decubitus ulcers may be greatly reduced owing to the age of the patient, the medical condition of the patient, smoking, or drugs.

With susceptible patients, it is frequently difficult to successfully prevent the development of decubitus ulcers or to prevent the worsening of existing decubitus ulcers. Customary measures include pressure distribution by frequently changing the patient’s position or by providing the place to lie, or a wheelchair, with pressure-reducing mattresses or cushions.

The mechanisms leading to the development of decubitus ulcers are not fully understood. Since pressure and shearing forces acting on skin and tissue promote the development of decubitus ulcers, measures serving for the prophylaxis of the development or worsening of decubitus ulcers should aim at reducing these variables.

There is therefore a need to make products available that can be used for effective prevention of the development of decubitus ulcers. Furthermore, there is a need to make products available that can be used for prevention of the worsening of decubitus ulcers that have already developed.

The present invention according to claim 1 solves the aforementioned problems.

In the present application, the term “proximal surface” is understood to mean the side of a layer or a ply that faces the skin covered by the article, whereas the term “distal surface” refers to the side that faces away from the skin. Where below-described components of medical articles according to the invention have a sheetlike extent in the broadest sense, such components each have a proximal surface and a distal surface. These surfaces are delimited at their sides. Where the term “on all sides” is used in the context of the present application, it is to be understood to mean all sides delimiting a surface of a ply or layer.

A medical article according to the present invention comprises a compress and a skin-contact layer adhesive to skin to be treated. The compress comprises a core and a shell surrounding the core. The core comprises a nonwoven material composed of fibers and absorbent particles. The compress has a longitudinal direction having a first modulus of elasticity and a transverse direction having a second modulus of elasticity. At the same time, the first modulus of elasticity is greater than the second modulus of elasticity.

In a preferred embodiment, the shell comprises a first ply composed of a liquid-permeable material and arranged on the proximal surface of the core and a second ply composed of a material different from the material of the first ply of the shell and arranged on the distal surface of the core. At the same time, the first ply of the shell projects beyond the proximal surface of the core on all sides and the second ply of the shell projects beyond the distal surface of the core on all sides, so that the first ply of the shell and the second ply of the shell each form an edge region which surrounds the core on all sides. The first ply of the shell and the second ply of the shell are joined to one another in said edge region.

To clarify, it should be mentioned that the terms “longitudinal direction” and “transverse direction” are not to be understood as meaning that a medical article according to the invention has a greater longitudinal extent in one of the two directions than in the other direction. Medical articles according to the invention can also have a substantially square or circular shape.

In the context of the present invention, the anisotropy of a layer or ply is understood to mean that its mechanical properties are differently manifested depending on the measurement direction. Preferably, an anisotropic layer or ply in the context of the present invention exhibits anisotropy with respect to its modulus of elasticity. Preferably, a layer or ply has a longitudinal direction having a first modulus of elasticity and a transverse direction, perpendicular to the longitudinal direction, having a second modulus of elasticity, the first modulus of elasticity being greater than the second modulus of elasticity.

Preferably, the longitudinal direction of the compress corresponds to the direction perpendicular to the machine direction of the production process for the medical article, whereas the transverse direction corresponds to the direction parallel to the machine direction of the production process for the medical article.

The core has a proximal surface and a distal surface. The core comprises a nonwoven material composed of fibers and absorbent particles. The material can contain any type of wound exudate-absorbent material, especially absorbent fibers and absorbent particles. Such a material performs a dual function in the article according to the invention, since it can exhibit an advantageous cushioning effect owing to the fiber-containing structure and can make an advantageous contribution to improving the microclimate on the affected skin area owing to the absorbent particles. Moisture occurring because of cutaneous respiration, perspiration or the egress of wound exudate, for example, can thus be absorbed by the particles. As described above, the occurrence of liquid on the skin surface promotes an increase in the shearing forces between skin surface and a material in contact with the skin, which can promote the development of new decubitus ulcers or the worsening of existing decubitus ulcers. An article according to the invention counteracts this risk owing to the absorption of liquid.

The material suitably contains a blend of absorbent fibers and absorbent particles. In this connection, a nonwoven ply composed of fibers and absorbent particles particularly performs the stated dual function when the absorbent particles are distributed substantially uniformly in the nonwoven material composed of fibers. When liquid is absorbed, the absorbent particles swell and form gel-like structures which bond with the surrounding fibers and thus form a network of fibers and gel particles that are particularly suitable for deflecting pressure and shearing forces from the skin surface and the underlying tissue.

Suitable materials for absorbent fibers can be, for example, cellulose or cellulose-based polymers, viscose, polyester, polyamide, and derivatives, copolymers and mixtures thereof. Furthermore, fibers having superabsorbent properties and based on polyacrylic acid, sodium polyacrylate, polyacrylates, and derivatives, copolymers and mixtures thereof are suitable. A preferred material is cellulose.

Suitable materials for absorbent particles include cellulose derivatives, alginates, carboxymethylcellulose, and derivatives, copolymers, mixtures and salts thereof, and superabsorbent particles comprising polyacrylic acid, sodium polyacrylates, polyacrylates, and derivatives, copolymers and mixtures thereof.

In a preferred embodiment, the core of the compress comprises a blend of absorbent fibers and superabsorbent materials. Absorbent fibers achieve rapid absorption of wound exudate, whereas superabsorbent materials achieve a high absorption capacity. Suitable absorbent fibers can be cellulose-based materials. In a preferred embodiment, the absorbent fibers substantially consist of cellulose fibers. Suitable superabsorbent materials are in the form of particles.

In a preferred embodiment, the core of the compress contains a blend of absorbent fibers substantially consisting of cellulose and superabsorbent particles containing polyacrylic acid and sodium polyacrylate. Such a core achieves rapid absorption of moisture, in particular wound exudate, and rapid distribution of moisture within the core.

The superabsorbent material can be contained within the core of the compress in an amount from 1% by weight up to 99% by weight. Preferably, the amount of superabsorbent material is a proportion from 30% by weight to 55% by weight of the core of the compress.

The term “superabsorbent material” is basically understood to mean a water-insoluble, swellable polymer capable of absorbing and binding many times its own weight in liquid, such as water, saline solution or body fluids, for example wound exudate. The absorption of liquid results in the formation of a hydrogel. The absorption capacity for pure water is generally greater than for a saline solution or body fluid. In connection with the present invention, the term “superabsorbent material” is understood to mean a material having a w-value for the free swelling capacity in accordance with the standard measurement method WSP 240.2 (05) of at least 10 g/g, preferably at least 20 g/g. The measurement method WSP 240.2 (05) for determining the w-value is described in “Standard Test Methods for the Nonwovens and Related Industries”, 2008 edition (published by “EDANA, International Association Serving the Nonwovens and Related Industries”, Cary, N.C., USA and “INDA, Association of the Nonwoven Fabric Industry”, Brussels, Belgium).

In a preferred embodiment, the core of the compress has an absorption capacity of from 100 g/100 cm² to 5000 g/100 cm², preferably from 120 g/100 cm² to 4000 g/100 cm² and particularly preferably from more than 130 g/100 cm² to 3000 g/100 cm² in accordance with a test method described in example 4 of the present application.

The core is surrounded by a shell. A shell is understood to mean an element which completely surrounds the core. The shell can be formed from one or more plies. At the same time, individual plies can project beyond the surfaces of the core. The ply parts projecting beyond the core can be oriented in a sheetlike manner and thus form an edge region projecting beyond the core. Alternatively, the ply parts projecting beyond the core can be folded over an edge of the core material in the projecting region, so that the core material is wrapped by this section of material.

In a preferred embodiment, the shell comprises a first ply composed of a liquid-permeable material having hydrophilic properties. The material can comprise a material which has hydrophilic properties owing to its chemical nature or a material which has originally hydrophobic properties owing to its chemical nature and has been treated in a chemical or physical process such that its surface has hydrophilic properties. Suitable methods for hydrophilization of originally hydrophobic materials encompass electrochemical processes, flame treatment, corona treatment and plasma treatment.

Suitable materials for the first ply of the shell are cellulose, polyester, polyamide, polyethylene, polypropylene and copolymers of two or more of the aforementioned materials. Preferably, the first fly of a hydrophilic material comprises either a polypropylene nonwoven which has been physically treated such that its surface exhibits hydrophilic properties or a nonwoven material which contains 55% polyamide fibers and 45% viscose fibers having a basis weight of 37 g/m². Such a material can be obtained from Freudenberg (Germany) under the name M1526. As an alternative preference, a nonwoven comprising 63% polypropylene fibers and 37% viscose fibers having a basis weight of 45 g/m² can be obtained from Freudenberg (Germany) under the name 670532/2.

In a preferred embodiment, the shell comprises a second ply composed of a material having hydrophobic properties. The material can have hydrophobic properties owing to its chemical nature or contain a material which has originally hydrophilic properties, but which has been modified by chemical or physical treatment, for example by treatment with hydrofluorocarbons, silicones, alkanes, etc., such that it exhibits hydrophobic properties. Suitable base materials for the second ply of the shell are cellulose, polyester, polyamide, polyethylene, polypropylene and copolymers of two or more of the aforementioned materials.

In a preferred embodiment, the first ply of the shell comprises a first material having hydrophilic properties and the second ply of the shell comprises a second material having hydrophobic properties.

In a further preferred embodiment, the second ply of the shell comprises a substantially liquid-impermeable material.

The two plies of the shell surrounding the core on all sides can be of any color. Medical articles are customarily white in color, which signals purity and sterility. In a further embodiment, at least one of the two plies is light green in color. This color achieves a strong color contrast when the ply has been wetted with liquid, and so a nurse can immediately tell from looking externally at a medical article applied to the patient when the core is containing liquid or when the maximum absorption capacity of the article has been reached. The presence or amount of absorbed liquid within the core of an article according to the invention can clearly indicate that a decubitus ulcer has just developed in the region of the skin covered by the article or that the extent of a decubitus ulcer covered by the article has changed. It was found that, surprisingly, a green color corresponding to RAL6019 ensures a particularly advantageous color contrast with wound exudate while still being perceived as clean and sterile by patients and nursing staff.

The first ply and the second ply of the shell of the core are joined to one another. This joining can be achieved by any suitable process for joining materials, be it directly or indirectly.

In a preferred embodiment, both the first ply and the second ply of the shell each comprise a material having thermoplastic properties. It is thus possible to join the first ply and the second ply of the shell to one another in a thermal process step. Thermal joining processes can be carried out continuously and can thus be more cost-effective to use than other processes. Suitable thermal processes for joining materials include heat welding, laser welding and ultrasonic welding.

In a preferred embodiment, the second ply of the shell comprises a thermoplastic material having hydrophobic properties and the first ply of the shell comprises a thermoplastic material which is the same material as that of the second ply of the shell and has been treated in a chemical and/or physical process such that it has hydrophilic properties. If the first ply and the second ply comprise materials differing in chemical nature, the physical properties can significantly deviate from one another. This can have an adverse effect in a joining process, in which it is necessary to find conditions which must be compatible with all participating materials. In a thermal joining process, the operating temperature must be higher than the melting points or melting ranges of each individual participating material. However, thermolabile materials can be destroyed if the temperatures are too high. Therefore, the selection of suitable materials for the first ply and the second ply can be considerably affected depending on the chemical nature of the materials. If the first ply and the second ply comprise materials of the same chemical nature, said materials do not differ in their physical properties relevant to carrying out a joining process. This is particularly advantageous in the case of thermal joining processes when the process temperature is only slightly above the melting ranges of the participating materials. The risk of thermal destruction is thus considerably reduced.

In a preferred embodiment, the thermoplastic material of both the first ply and the second ply of the shell comprises polypropylene.

In a preferred embodiment, the first ply and the second ply of the shell surrounding the core have been joined to one another in a thermal process and therefore have a weld connection to one another. A weld connection from a thermal joining process has an advantageous connection owing to its weld strength and flexibility. In the context of the present invention, weld strength means the force required to separate the two plies joined to one another. Weld strength can be determined using a method described in the examples of this application. In a preferred embodiment, the weld strength is greater than 0.75 N/15 mm, preferably greater than 1 N/15 mm.

A high weld strength is advantageous for prevention of unintentional delamination of the materials joined to one another. Delamination of the first ply and the second ply of the shell, even to a slight extent, can lead to leaks within the article. In this case, what can occur within the article are additional shearing forces which further promote the development or worsening of decubitus ulcers.

In the context of the present invention, flexibility means the property which enables a material to change its conformation. It is described by the stiffness of the material.

Flexibility is important for a medical article for the purpose of conforming to a patient’s body surface, which is often irregular. Furthermore, inflexible dressings are very stiff and, when moved, place pressure and stress on a patient’s skin surface, which can cause pain and promote the development or worsening of decubitus ulcers.

In a preferred embodiment, the weld connection comprises at least one discontinuous weld line. A discontinuous weld line exhibits advantageous flexibility, which has a positive effect on wearing comfort for a patient and counteracts the development or worsening of decubitus ulcers.

In a preferred embodiment, the weld connection comprises four to six parallel discontinuous weld lines, which results in advantageous balancing of the properties weld strength and flexibility.

In a preferred embodiment, the at least one discontinuous weld line is oriented parallel to the machine direction in the production process.

The compress can also contain a diffusion layer. Such a layer allows faster distribution of moisture, in particular wound exudate, in the horizontal direction within the core of the compress. Said layer can be located on the proximal surface of the core between the proximal ply of the shell and the proximal surface of the core. The diffusion layer can also be a material ply which envelops the entire core. Preferably, a diffusion layer is a paper layer which completely envelops the core. In a preferred embodiment, said paper is made of cellulose and has a basis weight of from 15 to 20 g/m².

The skin-contact layer adhesive to skin to be treated comprises a material which comprises a skin-friendly adhesive. Suitable adhesives can be based on acrylates, acrylate esters, polyvinyl ethyl ethers, polyurethane, silicone, and derivatives, mixtures or copolymers thereof, as described in GB1280631 for example. Preference is given to a pressure-sensitive adhesive.

In a preferred embodiment, the skin-contact layer adhesive to skin to be treated comprises a ply of a skin-friendly silicone adhesive. The silicone adhesive is suitably a so-called soft skin adhesive silicone elastomer. The total coating mass of the silicone adhesive is suitably from 15 g/m² to 500 g/m², preferably 50 g/m² to 250 g/m², particularly preferably 100 g/m² to 200 g/m².

It was found that, surprisingly, such a medical article has advantageous properties with respect to adherence strength on skin. A method for determining adherence strength on skin is described in the examples of the present application. A suitable medical article must be able to adhere to skin without loss of adherence strength, so that use of the article for several hours can be ensured, preferably use for at least one day, particularly preferably use for at least three days, most preferably use for seven days. On the other hand, an article must have skin-friendly properties which enable removal of the article after it has been used, without causing pain or skin irritation to the patient. It was found that, surprisingly, an article having an adherence strength of 200 mN/cm, preferably 350 mN/cm and 650 mN/cm, is advantageous in this respect.

In a preferred embodiment, the skin-contact layer adhesive to skin to be treated has openings which allow passage of wound exudate to the core. Said openings can have any geometric shape, regular or irregular, in particular a circular, oval, triangular, square, pentagonal, hexagonal or other polygonal shape having a uniform or nonuniform side length.

In a preferred embodiment, the skin-contact layer adhesive to skin to be treated has openings of any shape having an average open area of from 0.03 mm² up to 7.0 mm².

In a preferred embodiment, the skin-contact layer adhesive to skin to be treated has openings, the total open area of which is between 10% and 25% of the total surface area of the skin-contact layer. It was found that, surprisingly, an open area in this range results in a preferred rate of absorption of wound exudate. In the context of the present invention, rate of absorption means the time required for absorption of wound exudate. It can be determined by a test method described in the examples of the present application. It was found that, surprisingly, the rate of absorption is particularly advantageous when the skin-contact layer adhesive to skin to be treated an open area of from 11% to 22%, preferably 12% to 18%. The density of the openings is suitably between 1000 to 1 000 000 openings per square meter, for example 5000 to 50 000 openings per square meter.

In a preferred embodiment, the skin-contact layer adhesive to skin to be treated comprises openings having a substantially circular shape, the average diameter of which is between 0.2 mm up to 3.0 mm. It was found that, surprisingly, the rate of absorption is advantageous when the skin-contact layer adhesive to skin to be treated has openings having a circular shape and an average diameter of from 2.2 mm to 2.8 mm

Although it is possible to provide a skin-contact layer adhesive to skin to be treated that substantially consists of a soft skin adhesive silicone elastomer, it is preferred when said skin-contact layer contains an additional ply of a material comprising openings corresponding to the openings in the layer of silicone adhesive without closure thereof. The material provided with openings can encompass any type of medically acceptable material, including textile materials such as nonwovens, weft-knitted fabrics, warp-knitted fabrics or woven fabrics. The material having openings is suitably a unitary material such as a polymer mesh or a perforated film. Suitable polymer materials encompass polyethylene, polypropylene, polyester, polyvinyl acetate, ethylene vinyl acetate and polyurethane. The ply material has suitably a thickness of from 1 µm to 100 µm, preferably 5 µm to 50 µm.

The skin-contact layer adhesive to skin to be treated can be provided with a layer of a medically acceptable adhesive on its distal side. Such a layer of adhesive ensures a particularly stable connection between the skin-contact layer adhesive to skin to be treated and the components of the article, in particular the compress and/or in the edge region of the outer ply, arranged on the distal side of the skin-contact layer adhesive to skin to be treated. Preferably, the adhesive is a pressure-sensitive acrylate adhesive.

A preferred ply material for the skin-contact layer adhesive to skin to be treated is a polyurethane material having a layer of silicone adhesive in an amount of 150 g/m², which is available from ASC (France) under the name Acrysil®.

In a preferred embodiment, an article according to the invention comprises an additional outer ply, wherein the outer ply comprises a water vapor-permeable and substantially liquid-impermeable film material.

The additional outer ply supports and stabilizes the compress and provides a barrier to microorganisms through the article. The additional outer ply is substantially liquid-impermeable, but permeable to water vapor. Preferably, the additional outer ply comprises a film material. The additional outer ply has a water vapor permeability of from 300 to 30 000 g/m²/24 h, preferably 1000 to 15 000 g/m²/24 h and, in a particularly preferred embodiment, 1000 to 5000 g/m²/24 h, measured in accordance with EN 13726. The additional outer ply has a thickness of from 10 µm to 500 µm, preferably from 15 µm to 300 µm, particularly preferably from 20 µm to 100 µm.

The additional outer ply can be of various geometric shapes, such as square, rectangular, round, oval, trapezoidal or polygonal, preferably with rounded corners.

The additional outer ply has a proximal surface and a distal surface. The distal surface preferably has low surface friction. The use of materials having low surface friction on the distal surface of the outer ply reduces shearing forces that can occur when contact is made with the surface of another object, for example a bed sheet, a mattress or a cushion, and can promote the development or worsening of pressure ulcers.

Suitable materials for the additional outer ply are polyurethanes, polyalkoxyalkyl acrylates and methyl acrylates, as disclosed in GB 1280631. The outer ply suitably comprises a continuous layer of a high-density polyurethane foam which predominantly comprises closed cells. A suitable material is a 30 µm thick polyurethane film, which is available from Coveris under the name 1305.

Preferably, the additional outer ply is transparent in order to make it possible to look at the compress. This is advantageous when assessing the condition of the skin areas covered by the article. In particular, what is thus possible is early detection of whether the condition of the skin areas under the article is worsening over the course of the treatment.

In a preferred embodiment, the proximal surface of the additional outer ply comprises a coating containing a pressure-sensitive adhesive and projects beyond the distal surface of the compress on all sides, so that it forms an adhesive edge region which surrounds the compress on all sides.

The adhesive secures the additional outer ply to further layers of the article, in particular to the compress and to the skin-contact layer adhesive to skin to be treated. The additional outer ply can be continuously or discontinuously/partially coated with adhesive on its proximal side. The additional outer ply can be continuously coated with adhesive, which means that the adhesive covers the entire proximal side of the additional outer ply with adhesive. In a further embodiment, the adhesive can be applied in the form of stripes, dots or in various patterns. In a further embodiment, the additional outer ply comprises a layer of adhesive on its proximal side, which layer of adhesive provides a region free of adhesive in the middle of the additional outer ply in order to reduce contact with the distal surface of the compress. Water vapor permeability in the region free of adhesive is thus increased. When the compress begins to swell as a result of absorption of moisture, its spatial extent changes. This can, on the one hand, lead to delamination due to shearing forces within the article. More significantly, however, the shearing forces that occur can exert stress on the skin covered by the article and thus promote the further development or worsening of decubitus ulcers. Furthermore, this has the advantage that the skin moisture contributing to the development of decubitus ulcers can be reduced. An article providing a region free of adhesive between distal surface of the compress and the proximal surface of the additional outer ply may be advantageous in this respect.

The adhesive is preferably a pressure-sensitive adhesive of a type customarily used for medical dressings. Suitable pressure-sensitive adhesives can be based on acrylates, acrylate esters, polyvinyl ethyl ethers, polyurethane silicone, and derivatives, mixtures or copolymers thereof, as described in GB1280631 for example. The basis weight of the amount of adhesive applied is suitably 10 g/m² to 100 g/m², preferably 15 g/m² to 50 g/m², particularly preferably 20 g/m² to 30 g/m².

It was found that, surprisingly, a medical article according to the invention has advantageous properties in the rate of absorption test described in the examples of this application.

It was found that, surprisingly, a medical article according to the invention has advantageous properties with respect to the balance between adherence strength on skin and rate of absorption.

The additional outer ply, the compress and the skin-contact layer adhesive to skin to be treated can have different extents or identical extents.

In a particular embodiment, the distal surface of the compress is covered by the additional outer ply. The additional outer ply has an extent greater in the two surface directions than the area of the distal surface of the compress, whereas the compress and the skin-contact layer adhesive to skin to be treated are coextensive. The outer ply therefore forms an edge area which completely surrounds the compress. This type of article is also referred to as an island-type.

In a particular embodiment, the distal surface of the compress is covered by the additional outer ply. The additional outer ply has an extent greater in the two surface directions than the area of the distal surface of the compress. The outer ply therefore forms an edge region which projects beyond the compress on all sides. The skin-contact layer adhesive to skin to be treated has an extent greater in the two surface directions than the area of the proximal surface of the compress. This skin-contact layer therefore forms an edge region which projects beyond the compress on all sides. At the same time, said skin-contact layer is coextensive with the outer ply. This type of article is also referred to as a sandwich-type.

The medical article, the additional outer ply, the core of the compress, the compress, the shell and the skin-contact layer adhesive to skin to be treated can each be of any suitable size and geometric shape, in particular squares, rectangles, circles, ovals, polygons. If the geometric shape of any of these plies has corners, said corners can preferably be rounded. This reduces the risk of curl-up of the article during use and delamination of further plies or the skin. In a preferred embodiment, the radius for rounded corners is 5 mm to 15 mm, preferably 12.5 mm.

A medical article according to the present invention can comprise at least one cover layer covering the adhesive regions on the proximal surface of the skin-contact layer adhesive to skin to be treated. The cover layer covers and protects the compress and prevents premature adherence of the adhesive portions of the article. A cover layer can comprise a film composed of polyethylene, polypropylene, hydrofluorocarbons, and paper coated with any of the materials stated or with silicone. A suitable material can be a 100 µm thick polyethylene film, which is available from Flextrus®.

A medical article according to the present invention can be applied to a skin area of a patient and fixed by means of the skin-contact layer adhesive to skin to be treated that is contained in the article, thus securing the skin area from contact with external materials, for example a bed sheet. The occurrence of shearing forces, which normally occur as a result of friction between the patient’s skin and an external material, is thus avoided. Since a medical article according to the present invention has a certain stiffness, the frictional force occurring between the outer ply of the article and an external material is not transmitted through the layers of the article to the skin surface covered by the article. Articles according to the present invention are therefore capable of reducing or even avoiding the occurrence of shearing forces on vulnerable skin areas. The frictional forces occurring between outer ply of the article and an external material can be estimated by determination of the coefficient of friction. A distinction is made between static coefficient of friction, which characterizes the adhesive friction between two materials, and dynamic coefficient of friction, which characterizes the sliding friction between two materials. It is important that both the static and the dynamic coefficient of friction do not exceed a certain value in order to minimize the risk of shearing forces occurring in the skin area covered by an article. Coefficients of friction are moisture-dependent. A skin surface wetted by sweat or exudate has greater sensitivity to the occurrence of harmful shearing forces than a dry skin surface. Equally, a crucial factor for the suitability of an article is whether the outer surface of the article retains its friction-reducing properties even under increased moisture. It was found that, surprisingly, both a static and a dynamic coefficient of friction of from 0.20 to 0.50, preferably from 0.25 to 0.45, is advantageous. Static and dynamic coefficients of friction can be determined by a method described in example 9 of the present application.

A medical article according to the present invention can also contribute to reducing the pressure exerted on a specific skin area and the underlying tissue. Pressure arises by the action of a force to a specific area, for example the weight of a body part on the skin surface in contact with an external material, for example a bed sheet, underneath the body part. In order for a medical article to be able to achieve a reduction in pressure, the force which can act on a skin area must be absorbed by the article and distributed over a larger area.

To this end, the article material must have a certain compressibility. Materials which are excessively highly compressible directly transmit the pressure acting on the surface in the direction of compression without providing for any significant distribution of the forces over a larger area. In contrast, materials of very little compressibility can only absorb little pressure. The compressibility of an article according to the invention can be determined in a method described in example 8 of the present application. Compressibility is defined by the extent of compression of the article at a given force. It also moisture-dependent. It was found that, surprisingly, an article exhibiting in the dry state under a load of 150 N a compression of from 2.0 to 5.0 mm, preferably 3.0 to 4.5 mm and particularly preferably 3.5 to 4.0 mm, while exhibiting in the wetted state under a load of 150 N a compression of from 2.0 to 6.0 mm, preferably 3.0 to 5.0 mm and particularly preferably 4.0 to 4.5 mm, is advantageous for preventing the development or worsening of decubitus ulcers.

A medical article also contributes to reducing the shearing forces that occur if it has different moduli of elasticity in different directions of extent. Modulus of elasticity is a material characteristic from materials engineering that, in the case of linear-elastic behavior, describes the proportional relationship between mechanical stress and strain during the deformation of a solid body. Mechanical stress is a measure of the internal stress of a body due to an external load, for example pressure and/or shearing forces. A material layer having a greater modulus of elasticity in a first direction than in a second direction perpendicular thereto can oppose a force acting parallel to the first direction and having a certain stability and yield in the direction perpendicular thereto. An article having a higher modulus of elasticity in a direction parallel to an external pressure-exerting force or a shearing force than in the direction perpendicular thereto can contribute to redirecting the forces that occur from their original force action from the skin covered by the article and the underlying tissue, and so the development or worsening of decubital ulcers can be reduced. What is crucial here is especially the ratio of the moduli of elasticity in different directions, i.e., the extent of the anisotropy. Modulus of elasticity can be determined in a cyclic tensile test described in example 7 of the present application. It was found that, surprisingly, an anisotropy of from 1.5 to 20.0, preferably from 2.0 to 10.0 and particularly preferably from 2.5 to 8.0 is advantageous for preventing the development or worsening of decubitus ulcers.

In a preferred embodiment, a medical article according to the invention comprises two or more plies having such an anisotropy. Here, each of the plies has a longitudinal direction having a first modulus of elasticity and a transverse direction, parallel to the longitudinal direction, having a second modulus of elasticity, the first modulus of elasticity being in each case greater than the second modulus of elasticity. In this case, the anisotropic effects of the individual plies can advantageously supplement one another synergistically if the two or more plies in the medical article are oriented such that the longitudinal direction of a first ply anisotropic in such a manner and the longitudinal direction of a second ply anisotropic in such a manner and optionally the longitudinal directions of further plies anisotropic in such a manner are oriented parallel to one another.

In a further embodiment, a medical article according to the invention comprises an element suitable for marking the material direction having a higher modulus of elasticity. The nature of such an element can be such that said element is recognized and understood by a user of the article through visual, acoustic or haptic perception. Such elements can be attached to any point on the article as long as the information is understandable. Preferably, such an element is attached on one of the external sides of the article. Elements which can be recognized by visual perception encompass, for example, color markings, geometric symbols, characters, lettering or combinations thereof. Elements which can be recognized by haptic perception encompass, for example, embossing, elevations or depressions of one or more of the materials used in the article that can, for example, be felt with the sense of touch. Marking of the different material directions can also be achieved by use in different directions of materials having different surfaces, the surface differences of which can be felt by the sense of touch. Particular preference is given to marking in the form of a visually or haptically perceptible arrow, the arrow direction of which is oriented along a preferred direction of application on the body surface to be treated. Particularly preferably, an article according to the invention has marking in the form of an arrow attached parallel to the longitudinal axis of the body of the patient to be treated, the arrowhead pointing in the direction of the patient’s head. In the case of application to parts of the body that cannot be unambiguously determined in relation to the longitudinal axis of the body of the patient, what is possible is marking of an article according to the invention in the form of an arrow, the arrowhead of which points in the direction of the action of pressure most likely to be expected.

Pressure-sensitive mats can provide information as to what pressures are occurring in a mattress below a skin surface. But they do not give any information about the pressure conditions within the tissue on which they are acting. A simulation using a finite element method (FEM) can be used to simulate and study the biomechanisms leading to the development or worsening of decubitus ulcers.

On the basis of an FEM simulation, it was found that, surprisingly, medical dressings according to the present invention are capable of preventing the development or worsening of decubitus ulcers when they are applied to skin areas frequently prone to developing decubitus ulcers.

A medical article as described above is therefore suitable for prophylaxis of the development of decubitus ulcers. In this case, the article is applied to a skin area which is still intact and which is particularly at risk of developing decubitus ulcers.

A medical article as described above is also suitable for use on skin areas at which a decubitus ulcer has already developed. Said areas can, inter alia, be distinguished by open skin wounds which release wound exudate to a greater or lesser extent. If treatment is inadequate, there is the risk with such decubitus ulcers that their condition will continue to worsen. In this case, the article is suitable for prophylaxis of the worsening of the decubitus ulcer that has already developed and for therapeutic treatment thereof. Since the article has both liquid-absorbing properties and pressure- and shearing force-reducing properties, an article according to the invention is particularly suitable for this use.

The present invention further encompasses an article according to the invention for use in the prophylaxis of the development of decubitus ulcers.

Furthermore, the present invention encompasses an article according to the invention for use in the prophylaxis of the worsening of decubitus ulcers that have already developed.

The present invention also encompasses an article according to the invention for use in the therapeutic treatment of a decubitus ulcer that has already developed.

The present invention also encompasses the use of a medical article according to the invention for production of a medical device for prophylaxis of the development of decubitus ulcers.

The present invention also encompasses the use of a medical article according to the invention for production of a medical device for prophylaxis of the worsening of decubitus ulcers that have already developed.

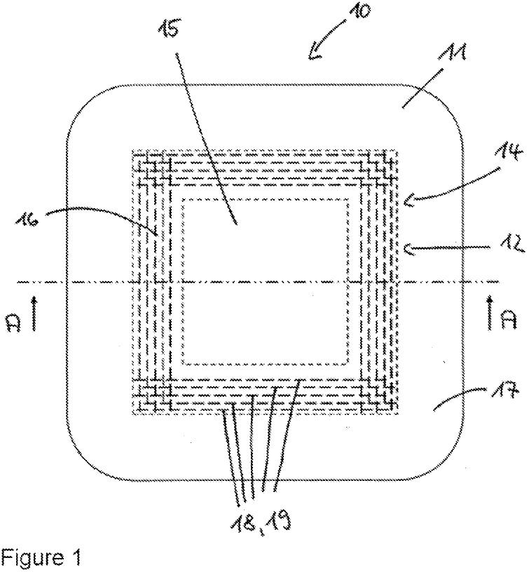

FIG. 1 shows a plan view of a medical article according to a preferred embodiment of the present invention.

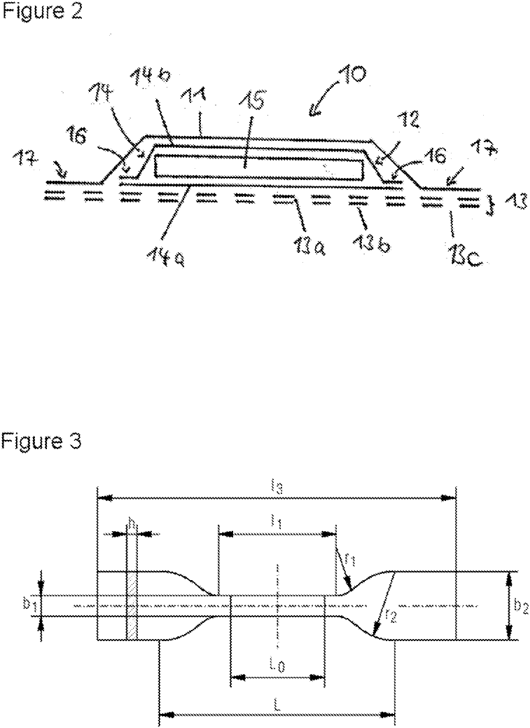

FIG. 2 shows a cross-sectional view of the article of FIG. 1 along the section line A-A of FIG. 1.

FIG. 3 shows a test specimen according to type 5A of DIN ISO 527 with its characteristic dimensions, as used for cyclic tensile tests according to example 7.

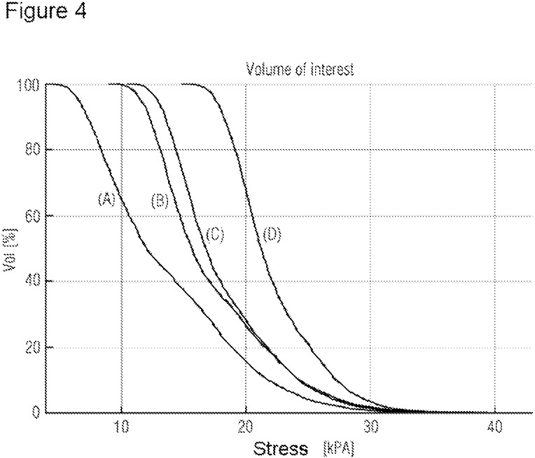

FIG. 4 shows a graph showing results of the FEM calculation. The abscissa shows von Mises stresses occurring on the skin. The ordinate shows the proportion of the relevant comparison volume (volume of interest, VOI) having von Mises stresses at least as great as the associated abscissa intercepts.

FIG. 1 shows a plan view of a medical article (10) according to the invention comprising an additional outer ply (11) and a compress (12). A skin-contact layer adhesive to skin to be treated is attached on the side facing away from the viewer. The compress (12) comprises a core (15) and a shell (14) surrounding the core (15). The shell (14) comprises a first proximal ply (facing away from the viewer) of a liquid-permeable nonwoven material and a second distal ply (14b) of a substantially liquid-impermeable nonwoven material. The proximal ply of the shell (14) covers the proximal side of the core (15) and projects beyond the proximal side of the core (15), with formation of an edge region (16) surrounding the core (15) on all sides. The distal ply (14b) of the shell (14) covers the distal side of the core (15) and projects beyond the distal side of the core, with formation of an edge region (16) surrounding the core (15) on all sides. The proximal ply of the shell (14) and the distal ply (14b) of the shell (14) have been joined to one another along the edge region (16) surrounding the core (15) by means of a thermal process and therefore have a weld connection (18). The weld connection (18) is formed by five discontinuous weld lines (19). The outer ply (11) projects beyond the compress (12), with formation of an edge region (17) surrounding the compress (12) on all sides.

FIG. 2 shows a cross-sectional view of the medical article (10) according to the invention of FIG. 1 along the section line A-A from FIG. 1, comprising a compress (12), an additional outer ply (11) and a skin-contact layer (13). The compress (12) comprises a core (15) and a shell (14). The shell (14) is formed from a proximal ply (14a) and a distal ply (14b). The skin-contact layer (13) is formed from a ply composed of a perforated sheet material (13a) and a ply of a skin-friendly silicone adhesive (13b). The outer ply (11) is formed from a water vapor-permeable and liquid-impermeable polyurethane film material having low frictional properties and having a thickness of 30 µm. The outer ply (11) is coated with a layer of an acrylatebased adhesive (not shown). The core (15) comprises a blend of cellulose fibers and superabsorbent sodium polyacrylate particles in a prefabricated airlaid material. The core (15) also comprises a diffusion layer (not shown) in the form of a ply of a cellulose paper wrapped around the nonwoven ply composed of a blend of cellulose fibers and polyacrylate particles. The proximal ply (14a) of the shell (14) is a nonwoven material composed of a mixture of viscose fibers and polyamide fibers. The distal ply (14b) of the shell (14) is a nonwoven material composed of polypropylene fibers. The proximal ply (14a) of the shell (14) covers the proximal side of the core (15) and projects beyond the proximal side of the core (15), said ply forming an edge region (16) surrounding the core (15) on all sides. The distal ply (14b) of the shell (14) covers the distal side of the core (15) and projects beyond the distal side of the core (15), with formation of an edge region (16) surrounding the compress (12) on all sides. The proximal ply (14a) of the shell (14) and the distal ply (14b) of the shell (14) are joined to one another along their edge region (16) surrounding the core (15). The perforated sheet material (13a) of the skin-contact layer (13) consists of a polyurethane film which has openings (13c) having a circular shape. Said openings (13c) have a uniform shape having an average diameter of 2.4 mm and are arranged in a regular pattern, resulting in a proportion of open area of 15%. The outer ply (11) projects beyond the compress (12). The skin-contact layer (13) projects beyond the compress (12), with formation of an edge region (17) surrounding the compress (12) on all sides. The outer ply (11) and the skin-contact layer (13) are coextensive.

FIG. 3 shows a test specimen, as used to determine the material properties in the tests according to example 7 (tensile strength), with the particular specified dimensions.

FIG. 4 shows a graph of the stresses (measured in kPa) occurring in the relevant model volume (volume of interest, VOI). The ordinate indicates what proportion of the relevant volume has a stress corresponding to at least the value on the abscissa that is associated with the respective curve point. Curve (A) shows the stresses which occur upon application of an object according to the invention as per example 1 to the skin in the relevant comparison volume. Curve (D) shows the stresses which occur in the relevant comparison volume without further protective measures. Curves (B) and (C) show the stresses which occur in the relevant comparison volume using two different competitor products.

### EXAMPLES

### Example 1 Medical Article

The medical article comprises a compress, an additional outer ply, and a skin-contact layer adhesive to skin to be treated. The compress comprises a core and a shell. The shell is formed by a proximal ply and a distal ply. The skin-contact layer is formed from a perforated sheet material and a ply of a skin-friendly silicone adhesive. The outer ply is formed from a water vapor-permeable and liquid-impermeable polyurethane film material having low frictional properties and a thickness of 30 µm.The core comprises a blend of cellulose fibers and superabsorbent sodium polyacrylate particles in a prefabricated airlaid material. The core also comprises a diffusion layer in the form of a ply of a cellulose paper wrapped around the airlaid blend of cellulose fibers and polyacrylate particles. The proximal ply of the shell is a nonwoven material composed of a blend of viscose fibers and polyamide fibers. The distal ply of the shell is a nonwoven material composed of polypropylene fibers. The perforated sheet material of the skin-contact layer consists of a polyurethane film which has openings having a circular shape. Said openings have a uniform shape having an average diameter of 2.4 mm and are arranged in a regular pattern such that the proportion of open area is 15%. The skin-contact layer additionally comprises a ply of a skin-friendly silicone adhesive without closing the openings of the perforated sheet material at the same time.

### Example 2 Test Solutions Used for Characterization of Medical Dressings

Solution A (saline solution)
2 L of deionized water
0.74 g of calcium chloride dihydrate (CaCl₂ ·2 H₂O, CAS: 10035-04-5)
16.6 g of sodium chloride (NaCl, CAS: 7647-14-5)

Solution B (exudate solution)
1 L of deionized water
70 g of albumin from chicken egg white (CAS: 9006-59-1)
0.2 g of Allura Red AC (CAS: 25956-17-6)
9 g of sodium chloride (NaCl, CAS: 7647-14-5)
0.37 g of calcium chloride dihydrate (CaCl₂ ·2 H₂O, CAS: 10035-04-5)
2 g of methyl 4-hydroxybenzoate (CAS: 99-76-3)
1 g of propyl 4-hydroxybenzoate (CAS: 94-13-3)

### Example 3 Rate of Absorption

The rate of absorption of an article is determined by the time required for complete absorption of a test liquid. Test solutions can be either saline solution (solution A) or exudate solution (solution B). Both the solutions and the test specimens must be preconditioned at room temperature before the test by storage of the test specimens and the solutions at 22° C. for two hours.

A 50 mL burette is filled with test solution. The liquid level is set at 15 mL. A test specimen is placed under the burette, the surface facing the burette being that which faces the skin when using the article. The distance between burette and test specimen is set at 1 cm. The burette tap is opened, while a stopwatch is simultaneously started. 2 mL of the test solution are allowed to flow out of the burette. The stopwatch is stopped once 2 mL of the test solution have been completely absorbed by the article, i.e., when there is no longer a drop of the test solution remaining on the perforation of the skin-contact layer. If the test specimen is sufficiently large, 1 to 3 measurements can be made on the same article. In this case, the test positions are in the center and two more points along a diagonal toward the corners of the article; they correspond to the positions of the spots of number 3 on a standard dice. At least five samples are tested.

Each value is classified in the following way:

| | | | | | |
|---|---|---|---|---|---|
| Time (s) | [0 to 4.9] | [5.0 to 10.9] | [11.0 to 30.9] | [31.0 to 60.0] | >60 |
| Category | Immediately | Very rapid | Rapid | Average | Slow |

An article according to the present invention was compared with the commercially available dressings Biatain® silicone, Allevyn® Life and Mepilex® border.

Rate of absorption

| Article | Rate of Absorption Rate [proportion of samples] | | | | |
|---|---|---|---|---|---|
| | Immediately | Very rapid | Rapid | Average | Slow |
| Example 1 | 20% | 80% | 0% | 0% | 0% |
| Biatain® silicone | 0% | 0% | 0% | 20% | 80% |
| Allevyn® Life | 0% | 0% | 100% | 0% | 0% |
| Mepilex® border | 0% | 0% | 0% | 100% | 0% |

### Example 4 Absorption Capacity

Both the solutions used (saline solution or exudate solution) and the test specimens must be preconditioned at room temperature before the test by storage thereof at a temperature of 22° C. for two hours.

The basic mass m₁ of an article is determined after removal of the protective layer covering the skin-contact layer. The length and width of the core of the compress is determined, so that the surface area S of the core of the compress can be determined. A bowl is filled with test liquid. The mass of the test solution should be at least 40 times greater than that of the article. The article is dipped into the bowl, while a stopwatch is simultaneously started. The side of the article that faces the skin in use should face the bottom of the bowl, and the reverse side of the article should be at the top. The article should not adhere to the bottom of the bowl. The article is left in the bowl for 30 min +/- 1 min. The dressings should only be touched at their edges and not at the core of the compress itself. The dressings are fixed in a clip at one corner and left hanging at room temperature for 20 min. The wet mass m₂ of the article is determined. The amount of absorbed test solution m_{liquid} is calculated as follows: m_{liquid} = m₂ - m₁

The absorption capacity is the quotient from the amount of absorbed test solution divided by the surface area S of the core of the compress, having the unit g/100 cm².

Absorption capacity = (m₂-m₁/S)*100

An article according to the present invention was with the commercially available dressings Biatain® silicone, Allevyn® Life and Mepilex® border.

Comparison of absorption capacities:

| Article | Absorption Capacity in g/100 cm² |
|---|---|
| Example 1 | 147 |
| Biatain® silicone | 116 |
| Allevyn® Life | 87 |
| Mepilex® border | 54 |

### Example 5 Adherence Power

Test subjects were informed about the purpose of the test and gave their written consent. Test products were applied to the back of each individual test subject (two samples per test product): One sample of each test product was secured on the top half of the back. The second sample was secured on the bottom half of the back.

The bottom end of each sample was folded over at a distance of 0.5 cm from the bottom edge in order to provide a starting point for pulling off the sample from bottom to top using a universal testing machine. The samples were applied by trained personnel and pressed onto the subject’s skin with the aid of a metal roller (1 kg, rolling back and forth five times).

The test subjects arrived at the study site 3 hours and 50 minutes after sample application and stayed in an air-conditioned room for at least 10 minutes. A check was made to determine that the subjects were not sweating. 4 hours after application of the samples, the samples were pulled off by means of a universal testing machine to determine the adherence power.

Universal testing machine: Zwick 1120 (Zwick GmbH, Ulm, Germany). The universal testing machine measures the force required to detach the samples from a test subject’s skin. To detach the samples, the test subjects were placed in a sitting position. The samples were pulled off at an angle of approximately 135°. One measurement was made for each sample, and two samples were tested from each product.

### Example 6 Weld Strength

Weld strength is determined using an MTS C42.503E tensile tester instrument (MTS Systems Corporation, Eden Prairie, USA) at a cell strength of 50 N.

Samples were prepared by punching out a portion of the core of the compress that contains a weld line and has a rectangular shape 15 mm wide and 25 mm long, the 15 mm side corresponding to the weld line. The sample must be taken at least 2 mm from the corner of the product. After the punch-out procedure was carried out, the core material was removed, leaving only a piece composed of two nonwoven materials joined to one another. The grips of the tester were set such that the distance between them was 2 cm. The two nonwoven pieces were clamped in separate grips. The tester was started at a speed of 200 mm/min until the two nonwoven pieces were separated from one another.

The weld strength is the average strength over the test period. It is reported in N/15 mm.

### Example 7 Tensile Strength

### Sampling

Test specimens corresponding to type 5a in accordance with DIN ISO 527, the shape of which is reproduced in FIG. 3, are punched out of the products tested. The dimensions correspond to the following values:

| | | |
|---|---|---|
| l₁ | Length of narrow parallel portion | 25 ± 1 |
| l₃ | Overall length | ≥ 75 |
| b₁ | Width of narrow portion | 4 ± 0.1 |
| b₂ | Width at ends | 12.5 ± 1 |
| r₁ | Small radius | 8 ± 1 |
| r₂ | Large radius | 12.5 ± 1 |
| L | Initial distance between grips | 50 ± 2 |
| L₀ | Gauge length | 20 ± 0.5 |

Test specimens were taken both in a direction parallel to the machine direction of the production process and in a direction perpendicular to the machine direction of the production process.

### Cyclic Tensile Testing

The mechanical properties of the punched samples were tested in a cyclic tensile test at a test temperature of 35° C. by means of a Z005 universal testing machine from Zwick/Roell with a temperature chamber. The samples were strained twice up to a nominal strain of 15% and then unloaded and subsequently tested to break.

### Test Procedure

Measuring device: Zwick/Roell Z005 (max. 5 kN)
Load cell: 500 N
Displacement transducer: Crosshead
Free clamping length of the sample: lo = 50 mm
Preload: 0.1 N (measurement begins after the preload has been reached (load = 0; displacement = 0))
Test speed until the preload is reached: 5 mm/min
Test speed: 50 mm/min
Number of loading and unloading cycles: 2 Ls
Cyclic loading: Up to 15% nominal strain
$$\left({ε}_{n o m} = \frac{Δ s}{{s}_{0}}\right)$$
s₀
Holding time at 15% nominal strain: 1 second
Measured values: Force F and crosshead displacement Δs
Test temperature: 35° C.
Test atmosphere: Air

### Preparation of the Test Specimens by Stamping

Sample: Waisted tensile sample type 5A (DIN EN ISO 527-2:2012-06) Sample width: 4.1 mm

### Measuring Device For

measuring thickness: HEIDENHAIN N221 measurement sensor

In the tensile test, the forces and deformation displacements were measured and presented as force-displacement graphs for the various samples tested.

The modulus of elasticity is defined as the slope of the graph in the stress-strain graph:

Modulus of elasticity E = σ / ε

with stress σ and strain ε.

Here, σ = F / A refers to the mechanical stress force per cross-sectional area of the test specimen.

Here, ε = Δl/ l₀ with change in length Δl = l - l₀ refers to the strain, I referring to the length of the test specimen after the tensile test and l₀ referring to the initial length of the test specimen.

What were tested were both test specimens that were taken in a direction parallel to the machine direction of the production process and test specimens that were taken in a direction perpendicular to the machine direction of the production process. The moduli of elasticity thereof were ratioed with one another to calculate the anisotropy of the respective materials.

The following table lists the thus determined anisotropies with respect to the moduli of elasticity of the individual components of a medical article according to example 1:

| Ply | Anisotropy |
|---|---|
| Outer ply | 1.1 |
| Compress | 2.5 |
| Nonwoven material | 3.7 |
| Distal ply of the shell | 2.4 |
| Skin-contact layer | 0.9 |

### Example 8 Compression

### Cyclic Compression Test

The mechanical properties of the products were tested in the dry state and in the waterwetted state in a cyclic compression test at a test temperature of 35° C. by means of a Z005 universal testing machine from Zwick/Roell with a temperature chamber. The samples were compressed twice up to a load of 150 N and then unloaded and subsequently tested up to a maximum of 450 N.

### Test Procedure

Measuring device: Zwick/Roell ZOOS (max. 5 kN)
Load cell: 500 N
Displacement transducer: Crosshead
Preload: 0.1 N (measurement begins after the preload has been reached (load = 0; displacement = 0))
Test speed until the preload is reached: 2 mm/min
Test speed: 2 mm/min
Number of loading and unloading cycles: 2
Cyclic loading: Up to 150 N load
Holding time at 150 N: None
Measured values: Force F and crosshead displacement Ds Distance between the compression platens at preload (= initial gauge length)
Compression platens: Steel, dry (diameter = 60 mm)
Test temperature: 35° C.
Test atmosphere: Air

### Preparation of the Test Specimens

Sample: Patch was adhesively bonded on lower compression platen at the center and in a dry or wet (wetted with water) state Wetting: Distilled water (0.15 mL/cm²) - an amount of water corresponding to the sample area was applied to the product surface facing away from the skin using a pipette and allowed to soak in for one hour, after which the test was carried out.

### Example 9 Coefficient of Friction

### Friction Tests

Using a rotary tribometer, the samples were tested in the dry and wetted state for their frictional behavior in relation to a cotton surface. Wetted means that the sample has been wetted at the surface on the top layer using 0.15 ml/cm² distilled water. An amount of water corresponding to the sample area was applied to the patch surface using a pipette and allowed to soak in for one hour, after which the test was carried out. To this end, the sample is adhesively bonded to a stationary, flat steel surface (diameter 60 mm). The rotating counter partner is a cotton fabric which has been adhesively bonded to a steel stamp (diameter 60 mm) by means of double-sided adhesive tape. The sample and the counter surface are pressed against one another with a contact force of 50 N, and then the coefficient of friction between sample and rotating counter surface is measured at different sliding speeds.

The drive motor rotates at different defined rotational speeds corresponding to a sliding speed of from 1 mm/s to 100 mm/s. The sliding speed is calculated on the basis of the average diameter of 30 mm.

### Test Procedure

Measuring device: Self-made rotary tribometer
Force-torque sensor: Max. 100 N
Preload: 50 N (static) corresponds to normal load
Stamp diameter of the friction contact surfaces: 60 mm
Counter partner: Cotton
Sliding speed v for average stamp diameter d = 30 mm: Variable, 1 mm/s to 100 mm/s
Rotational speed n: Variable depending on sliding speed,
$$n = \frac{v}{π ⋅ d} ⋅ 60$$
Speed profile: 15 levels, see table
Number of samples: 3 each
Test temperature: 23° C. (room temperature)
Test atmosphere: Air

Test conditions of the friction tests

| Level | Sliding speed v for 30 mm diameter [mm/s] | Rotational speed n [rpm] | Time span Δt [s] | Time t [s] |
|---|---|---|---|---|
| 1 | 0.0 | 0.00 | 5 | 5 |
| 2 | 0.2 | 0.13 | 5 | 10 |
| 3 | 0.0 | 0.00 | 5 | 15 |
| 4 | 1.0 | 0.64 | 100 | 115 |
| 5 | 5.0 | 3.18 | 20 | 135 |
| 6 | 10 | 6.37 | 10 | 145 |
| 7 | 20 | 12.74 | 10 | 155 |
| 8 | 30 | 19.11 | 10 | 165 |
| 9 | 40 | 25.48 | 10 | 175 |
| 10 | 50 | 31.85 | 10 | 185 |
| 11 | 60 | 38.22 | 10 | 195 |
| 12 | 70 | 44.59 | 10 | 205 |
| 13 | 80 | 50.96 | 10 | 215 |
| 14 | 90 | 57.32 | 10 | 225 |
| 15 | 100 | 63.69 | 10 | 235 |

The medical dressings tested had the following coefficients of friction (COF): Adhesive friction (dry) COF_{dry} = 0.36 Adhesive friction (wetted) COF_{wetted} = 0.41 Sliding friction (dry) COF_{dry} = 0.28 Sliding friction (wetted) COF_{wet} = 0.36

### Example 10 Finite Element Method (FEM)

The FEM is a general and computer-aided numerical method used for different physical problems. The FEM is logically based on numerically solving a complex system of differential equations. The FEM divides large problems into a multitude of smaller parts called finite elements. Analysis is carried out with each of these elements and, taken as a whole, results in a solution for the entire problem.

The work steps of an FEM can be described as follows:
1. creation of a 2D or 3D model consisting of finite elements;
2. definition of the material properties of the model;
3. definition of the boundary conditions and loads for application of the model to the problem;
4. computer-aided solving of the problem; and
5. analysis of the results through visualization and calculation.

The FEM calculation underlying the invention was done according to the method described in the following article: Levy A, Schwartz D, Gefen A. The contribution of a directional preference of stiffness to the efficacy of prophylactic sacral dressings in protecting healthy and diabetic tissues from pressure injury: computational modelling studies. Int Wound J 2017; doi: 10.111/iwj.12821

To understand the effects of medical dressings according to the present invention, FE models of a human pelvis and the dressings were created. The influences of pressure and stress on the skin and deeper tissues were analyzed.

The pelvic model was based on MRI scans of a voluntary female test subject in order to ensure the greatest possible anatomical accuracy of the model.

The FE models comprise 3 900 000 nodes.

Soft tissues were represented as nonlinear materials, with muscles being amalgamated to form one material and fat and skin each being amalgamated as compressible materials. The bones were amalgamated as a rigid body.

Modeling was based on the following material properties:

Bed: Linear modulus of elasticity E = 50 kPa Bone: Linear modulus of elasticity E = 7000 MPa Adipose tissue: Hyperelasticity (neo-Hooke) C10 = 0.0004 Muscle tissue: Hyperelasticity (neo-Hooke) C10 = 0.000225 Skin: Hyperelasticity (neo-Hooke) C10 = 0.004

A relevant model volume (volume of interest, VOI) having dimensions of 6.7 cm x 2.0 cm x 5.1 cm (x-direction x y-direction x z-direction) was formed, containing the sacrum (os sacrum) and the surrounding soft tissue including skin.

Von Mises stress refers to a fictitious uniaxial stress which, on the basis of a specific material-mechanical or mathematical criterion, represents a hypothetically equivalent material stress, such as a real multiaxial state of stress.

On the basis of the von Mises stress, the real, generally three-dimensional state of stress in the component in the strength or yield condition can be compared with the characteristics from the uniaxial tensile test (material characteristics, for example yield point or tensile strength).

The von Mises stresses can be calculated according to the following formula:

$${σ}_{v , M} = \sqrt{\frac{1}{2} \left[{\left({σ}_{I} - {σ}_{I I}\right)}^{2} + {\left({σ}_{I I} - {σ}_{I I I}\right)}^{2} {\left({σ}_{I I I} - {σ}_{I}\right)}^{2}\right]}$$

Here, σₗ, σₗₗ, and σₗₗₗ are the principal stresses occurring in the three spatial directions.

What are of particular importance are the stresses occurring on the skin within the relevant model volume, because what occur here are not only the pressures responsible for the development and worsening of decubitus ulcers, but also shearing forces.

A comparison was made in each case between calculations in which a medical article according to the invention was placed on a skin area with the same skin area without an applied medical article.

What is of significant interest is the 10% value. This value indicates what maximum stresses occur in not more than 10% of the relevant comparison volume. It corresponds to the curve point associated with the ordinate intercept at 10% VOI.

From the comparison of the von Mises stresses on the skin in the relevant model volume with and without an applied medical article, suitability for prevention of the development of decubitus ulcers can be postulated if the 10% value for the stresses that occur is reduced by more than 10%.

The curves shown in FIG. 4 have the following 10% values:

| | | |
|---|---|---|
| Curve (A): | 21.8239 kPa | Article according to the invention |
| Curve (B): | 24.3311 kPa | Competitor product 1 |
| Curve (C): | 24.2884 kPa | Competitor product 2 |
| Curve (D): | 27.2725 kPa | No article applied |

As can be seen from FIG. 4, the 10% value for the von Mises stresses that occur on the skin in the relevant model volume was reduced by 20% by use of a medical article according to the invention.

1. A medical article (10) for prophylaxis of the development and/or worsening of decubitus ulcers, comprising a compress (12) and a skin-contact layer (13) adhesive to skin to be treated, wherein the compress (12) has a proximal surface and a distal surface and comprises a core (15) and a shell (14) surrounding the core (15), wherein the core (15) has a proximal surface and a distal surface and comprises a nonwoven material composed of fibers and absorbent particles, wherein the compress (12) has a longitudinal direction having a first modulus of elasticity and a transverse direction having a second modulus of elasticity and the first modulus of elasticity is greater than the second modulus of elasticity.

2. The medical article (10) of claim 1, wherein the shell (14) comprises a first ply composed of a liquid-permeable material arranged on the proximal surface of the core (15) and a second ply composed of a material different from the material of the first ply of the shell (14) arranged on the distal surface of the core (15), wherein the first ply of the shell (14) projects beyond the proximal surface of the core (15) on all sides and the second ply of the shell (14) projects beyond the distal surface of the core (15) on all sides, and wherein the first ply of the shell (14) and the second ply of the shell (14) each form an edge region (16) which surrounds the core (15) on all sides, wherein the first ply of the shell (14) and the second ply of the shell (14) are joined to one another in said edge region (16).

3. The medical article (10) of claim 1, wherein the core (15) has an absorption capacity of from 100 g/100 cm² to 5000 g/100 cm².

4. The medical article (10) of claim 2, wherein the first ply of the shell (14) comprises a first material having hydrophilic properties and in that the second ply of the shell (14) comprises a second material having hydrophobic properties.

5. The medical article (10) claim 2, wherein both the first ply and the second ply of the shell (14) each comprise a material having thermoplastic properties.

6. The medical article (10) of claim 5, wherein the second ply of the shell (14) comprises a thermoplastic material having hydrophobic properties and the first ply of the shell (14) comprises a thermoplastic material which is the same material as that of the second ply of the shell (14) and has been treated in a chemical and/or physical process such that it has hydrophilic properties.

7. The medical article (10) of claim 5, wherein the first ply of the shell (14) and the second ply of the shell have been joined to one another in a thermal process and therefore have a weld connection (18) to one another.

8. The medical article (10) of claim 7, wherein the weld connection (18) comprises at least one discontinuous weld line (19).

9. The medical article (10) of claim 8, wherein the weld connection (18) comprises four to six parallel discontinuous weld lines (19).

10. The medical article (10) of claim 8, wherein the at least one discontinuous weld line (19) is oriented parallel to the machine direction in the production process.

11. The medical article (10) of claim 1, wherein the skin-contact layer (13) adhesive to skin to be treated comprises a ply of a skin-friendly silicone adhesive (13b).

12. The medical article (10) of claim 1, wherein the skin-contact layer (13) has openings, the total open area of which is between 10% and 25% of the total surface area of the skin-contact layer (13).

13. The medical article (10) of claim 1, wherein the skin-contact layer (13) has openings (13c) having a substantially circular shape, the average diameter of which is between 0.2 mm to 3.0 mm.

14. The medical article (10) of claim 1, wherein the article comprises an additional outer ply (11) having a proximal and a distal surface, wherein the additional outer ply (11) comprises a water vapor-permeable and substantially liquid-impermeable film material.

15. The medical article (10) of claim 1, wherein the proximal surface of the additional outer ply (11) comprises a coating containing a pressure-sensitive adhesive and the additional outer ply (11) projects beyond the distal surface of the compress (12) on all sides, so that it forms an adhesive edge region (17) which surrounds the compress (12) on all sides.

16. The medical article (10) of claim 1, wherein the additional outer ply (11) projects beyond the distal surface of the compress (12) on all sides and the skin-contact layer (13) is coextensive with the outer ply (11).

17. The medical article (10) of claim 1, wherein the article (10) comprises an element suitable for marking the material direction having a higher modulus of elasticity.

18. A method for the prophylaxis of the development and/or worsening of decubitus ulcers comprising employing the medical article (10) of claim 1.

19. A method for production of a medical device for prophylaxis of the development and/or worsening of decubitus ulcers comprising employing the medical article (10) of claim 1.
